(19) United States
(12) Patent Application Publication
(10) Pub. No.: US 2008/0131471 A1
(43) Pub. Date: Jun. 5, 2008

(54) Method of Transferring Bacteriostatic Properties to a Product in an Aqueous Solution

(75) Inventors: Axel Kolbe, Neundorf (DE); Hardy Markwitz, Rudolstadt (DE)

Correspondence Address: EDELL, SHAPIRO & FINNAN, LLC, 1901 RESEARCH BOULEVARD, SUITE 400, ROCKVILLE, MD, 20850, US

(73) Assignee: SMART FIBER AG, Rudolstadt (DE)

(21) Appl. No.: 11/947,076

(22) Filed: Nov. 29, 2007

(30) Foreign Application Priority Data
Nov. 30, 2006 (DE) 102006056977.6-41

(51) Int. Cl. A61K 33/38; A61K 9/00

(52) U.S. Cl. 424/404; 424/618

(57) ABSTRACT

Textiles and also dishes and other household implements which are cleaned in laundry machines or dish washing machines can be afforded protection from an increasing colonization by bacteria when the textiles or the dishes are washed together with a microbicidal repository including a solid fiber composite of ion-exchanger particles loaded with silver ions, and a cellulose matrix.

### CROSS REFERENCE TO RELATED APPLICATIONS

This application claims priority under 35 U.S.C. §119 to Application No. DE 102006056977.6-41 filed on Nov. 30, 2006, entitled “Use of a Textile Microbicidal Repository,” the disclosure of which is hereby incorporated by reference in its entirety.

### FIELD OF THE INVENTION

The present invention relates to providing a product with bacteriostatic properties and, in particular, to a process of releasing silver ions from a textile material including a microbicidal repository into an aqueous solution and then transferring the released ions to another item present in the solution.

### BACKGROUND OF THE INVENTION

There is an increasing interest in articles offering qualities of improved hygiene and freshness (e.g., clothing and clothes washing machines). For example, attempts have been made to provide washing machines with an anti-microbial agent to inhibit the growth of bacteria in the machine. One approach includes the mechanical coupling of a silver ion source to a washing machine. For use in laundry machines, different dosages of silver have been described. JP 2005261830A describes an electrical silver ion generator for enriching the washing water with silver ions. The silver ion generator is installed at the midstream of a water feeding system to a washing machine, and includes a power source independent of the power source of the washing machine. Utilizing the silver ion water generator reduces the consumption and consequently extends the life span of a cartridge for generating silver ions. This system suffers from several drawbacks. First, the positioning of the generator—between the water supply and the laundry machine—is only effective when rain water or germ-infested well water is used as the water source. Drinking water, by comparison, is substantially free of germs; consequently, an infestation of a laundry machine by germs typically occurs during long intervals between operations (and not during use). Second, the system requires the use of an independent power source.

Another way is to use nano silver (a colloidal suspension of silver), wherein nano sized silver particles are dispersed in water. For example, KR1020040093956A discusses a washing ball including nano silver formed by pulverizing silver into a fine powder and mixing it with synthetic resin. The surface area of the silver is increased by the utilizing extremely small grains, with the effective silver ions formed via surface oxidation. The washing ball includes a series of friction projections formed on its surface to increase friction with the laundry. In operation, the ball is placed into a laundry tub, and the washing machine cycle is initiated. During the cycle, nano silver is released into the tub, coating the tub with the fine powders of the silver to remove bacteria or molds stuck on the laundry. Similar nano silver mechanisms are disclosed in KR1020050114811A, KR1020040093958A, and KR1020040086672A. The nano silver process suffers from the disadvantage of a lack of control of the equilibrium concentration of the silver ions in the washing water. In addition, commercially, the conversion of silver to nano particles requires outlay and is costly.

Another approach, discussed in JP5111595, is to coat or embed the components of a washing machine with an antibacterial agent including an ion source, a carrier, a ceramic material which adsorb and carry the antibacterial ion source, and zinc oxide whiskers. The components are all fabricated from resin through a molding process, and the agent in the form of composite resin pellets is included in these resin components. With this mechanism, the antibacterial effect is associated only with the constructional components of the laundry machine, being unable to transfer the ions to items in the laundry.

In another approach, textile products may be made with fibers having antibacterial or fungicidal properties. For example, DE10140772 (US2005/0035057) discusses a method of removing heavy metals from media containing heavy metals, using a lyocell molded body to adsorb the heavy metals. Similarly, DE10315749 A1 (US2006283567) discusses a method wherein a fiber is incorporated with a binder formed from a weakly-cross-linked cationic exchanger. The binder binds bactericidal metal ions and/or ionic pharmaceutically active substances. The fibers are used to form sanitary textiles such as food packaging, bandages, and garments.

Other textile articles with antibacterial properties and methods of forming the articles are discussed in AT413818 (US2006/246285), US2006/0171996, WO2006/013378 (US2007/243380), and DE102005002539A (EP1655409).

Many organic anti-microbial agents have been used or proposed for use on fibers, including triclosan, biguanides, phenols and derivatives, isothiazolones, quaternary ammonium salts, tri-butyl tin oxide, haloamines, and alcohols. The most widely used of these is triclosan, which has been used as a fiber finish and fabric finish for both natural and man-made fibers and has also been incorporated into man-made fibers such as regenerated cellulose fibers and acrylic fibers by inclusion in the spinning dope.

Inorganic anti-microbial agents have also been used, and these are predominantly compounds in which a metal ion supported on an inert matrix. Heavy metal ions, such as silver, mercury, copper, zinc, and zirconium ions have a lethal or growth inhibiting effect on microorganisms such as bacteria, viruses, fungi and spores. For example, silver ions have a bactericidal effect. Silver ions, moreover, possess the benefit of being generally insensitive to the human metabolism, unlike mercury ions (Hg²⁺).

### SUMMARY OF THE INVENTION

A textile material includes an ion-exchanger, a hydrophilic binder, and bacteriostatic agent such as silver ions. The material is placed into an aqueous solution and the equilibrium concentration of the silver ions is maintained in a biologically effective (antimicrobial-effective) range. A product such as a woven article of clothing or a flower is placed into the aqueous solution in the presence of the textile material. The released ions from the textile material attach to a surface of the product, thereby providing the product with bacteriostatic properties.

### DETAILED DESCRIPTION OF THE INVENTION

The invention is directed toward providing products such as textiles, dishes, and other articles of use, or biological material such as, for example, cut flowers, with improved protection from being colonized and/or overgrown by bacteria.

The textile material includes a solid fiber composite of ion-exchanger particles loaded with silver ions and a hydrophilic matrix/binder. This essentially creates a microbicidal repository formed of a depot of silver ions. Through the use of ion-exchange particles based on polyacrylate in a solid composite with a hydrophilic matrix, silver ions can be deposited onto fiber in concentrations of up to about 10% w/w. The hydrophilic matrix may include cellulose. Through the use of cellulose as a hydrophilic, network-forming polymer, all ion-exchanger particles, whether inside or on the surface of the fiber, are fully accessible for ion exchange during a washing operation. The equilibrium concentration of the silver ions attains the biologically effective range. Owing to the strong interaction of the ion-exchanger polymer with the silver ions, the silver ions are given off slowly during the course of many washing operations. After 100 washing operations at about 60° C., the presence of about 46% of the silver depot was still present within the fibers, evidencing the textile repository was still biologically effective.

The fibers may include lyocell fibers. Lyocell fibers are produced by extrusion of a solution of cellulose through a spinning jet into a coagulation bath by a process known as solvent spinning. They are therefore alternatively known as solvent-spun cellulose fibers. Such a process is described in U.S. Pat. No. 4,246,221 and uses as the solvent an aqueous tertiary amine N-oxide, particularly N-methylmorpholine N-oxide. Lyocell fibers are distinguished from regenerated cellulose fibers, such as viscose fibers, which are produced by forming the cellulose into a soluble chemical derivative and then extruding a solution of this derivative into a bath, which regenerates the extrudate as cellulose fibers.

Surprisingly, and surpassing present prior art, it could be proved that the silver ions are deposited in smallest of concentrations on the surface of the textiles, and that the bactericidal effect extends beyond the actual washing step to a reduction of the germ count in washing water and on the textile material. The use of about 0.01-100 g, preferably about 5 g, of fibers directly with the goods to be washed is sufficient for this effect. The fibers may be added as component parts of a non-woven fabric, warp-knitted fabric, or woven fabric. The textile repository material may further be enveloped by a water-permeable synthetic resin or textile sheath. Generally, structures are possible which enable access of water to the supporting fiber, wherein permeable containers of glass, ceramics, metal, or natural products (e.g., bast fiber, wood, horn, bone, tortoise shell, and chitin), minerals, and semi-precious stones may also be utilized.

Also surprisingly, it has been found that the silver ions are transferred in a rapid process from the textile repository material via the aqueous phase to attach onto product surfaces (e.g., textile surfaces), and to impart the product with bacteriostatic/microbicidal (germ-inhibiting) properties. This effect may be used to great advantage during laundering by machine in a washing machine, and similarly during laundering by hand. A use of the textile repository material in a dish washer (washing of dishes by machine) or in a sink (manual dish washing) also transfers silver ions onto the cleaned articles, and renders difficult any renewed colonization by bacteria. In general, to be effective as bactericide, the concentration of silver should be present in an amount of 0.01-1 mg/l.

The biocidal or bacteriostatic effect also occurs with biological materials such as plants and flowers. It is particularly effective with cut flowers. Silver ions transferred from the textile repository material via the aqueous phase attach onto the surfaces of stalks of cut flowers and render difficult any colonization by bacteria which owing to their metabolism decompose the water-conducting passages and cause the plant to wither prematurely.

Thus, a product may be provided with a bacteriostatic property by placing the product and a textile microbicidal repository in an aqueous solution for a predetermined period of time (e.g., a time sufficient to permit the transfer of a biologically effective amount of microbicidal (silver ions) to the product). The product may include an article of clothing, a dish, a flower, etc. The textile microbicidal repository material may include fibers and a solid composite of silver-ion loaded ion-exchanger particles with a hydrophilic matrix. Placing the textile material into an aqueous solution causes the release of at least a portion of the silver ions from the textile material. During the predetermined time period, some or all of the silver ions in solution are transferred from the textile material to the surface of the product to provide the product with a bacteriostatic property. The aqueous solution may include a container of water (e.g., a flower vase), or may include a water-agitation device such as a dishwashing machine or a laundry machine.

The following Examples will serve to further elucidate the invention and the properties essential to the use. The used mineral, “SMARTCEL bioactive”, includes lyocell cellulose fibers with proportions of ion-exchanger-bound silver ions, and is manufactured chiefly according to the amine-oxide method as discussed in U.S. Pat. No. 4,246,221 and EP 1358371A. SMARTCEL bioactive is available from Smartfiber AG, Rudolstadt, Del.

### EXAMPLE 1

12.5 g of non-woven fabric containing a proportion of 0.625 g SMARTCEL bioactive were subjected to standard industrial washing together with a woven cotton fabric. For comparison, the washing test was repeated without a SMARTCEL bioactive non-woven fabric. Germ growth values for 18 hours were determined for the test germ Staphylococcus aureus ATCC 6538. The results are summarized in Table 1.

TABLE 1
Transfer of an antimicrobial effect from a non-woven fabric to
a cotton woven fabric

| | Cotton woven fabric | Cotton woven fabric | |
|---|---|---|---|
| | washed with | washed without | |
| | non-woven | non-woven | Control |
| | fabric | fabric | Polyester |
| Germ count 0 h | 2.23 · 10⁵ | 2.23 · 10⁵ | 2.23 · 10⁵ |
| Germ count 18 h | 1.07 · 10³ | 9.59 · 10⁵ | 2.48 · 10⁵ |
| Total activity | 2.36 | −0.59 | — |
| Assessment | Significant | No antimicrobial | |
| | antimicrobial | activity | |
| | activity | | |

### EXAMPLE 2

A pad consisting of 5 g SMARTCEL bioactive fibers sewn into a woven fabric was subjected to 100 washing cycles at 60° C. under normal conditions. Subsequently, a woven cotton fabric was washed together with the pad, and another woven cotton fabric without a pad. The two test samples were tested for specific antimicrobial activity against Staphylococcus aureus ATCC 6538 and Klebsiella pneumoniae ATCC 4352. The results are summarized in Table 2.

TABLE 2
Transfer of an antimicrobial effect from a SMARTCEL bioactive
pad to a cotton woven fabric under long-time conditions

| | | |
|---|---|---|
| Cotton Woven | Cotton Woven | |
| Fabric Washed | Fabric Washed | Control |
| With Pad | Without Pad | Polyester |

Staphylococcus aureus

| | | | |
|---|---|---|---|
| Germ count 0 h | 3.64 · 10⁴ | 3.64 · 10⁴ | 1.06 · 10⁵ |
| Germ count 18 h | 1.47 · 10³ | 2.35 · 10⁶ | 3.13 · 10⁵ |
| Assessment | Strong specific | No antimicrobial | |
| | antimicrobial activity | activity | |

Klebsiella pneumoniae

| | | | |
|---|---|---|---|
| Germ count 0 h | 9.01 · 10⁴ | 9.01 · 10⁴ | 1.04 · 10⁵ |
| Germ count 18 h | 1.29 · 10⁴ | 2.58 · 10⁷ | 1.89 · 10⁷ |
| Assessment | Strong specific | No antimicrobial | |
| | antimicrobial activity | activity | |

After 100 washing operations, 126 mg silver/pad was ere still detectable by analysis in the pads, which corresponds to about 46% of the initial value. After 80 washing operations, this value still amounted to about 53% of the initial value. This reduction corresponds to the equilibrium state. It can be supposed that approx. 1 mg of silver ions is given off by the pad per washing operation. According to the performed tests, this quantity is sufficient to render a normal textile antimicrobial. The pad can be calculated to be usable for at least 220 washing cycles before the effect in accordance with the invention becomes exhausted.

### EXAMPLE 3

A non-woven fabric having a weight of 0.5 g and a proportion of 0.05 g of silver-containing textile repository material (SMARTCEL bioactive) was added to a water-filled flower vase with fresh cut flowers. By comparison with a reference sample without pad, the cut flowers remained fresh on average for three days longer (the criterion being a visible withering and drooping of the blossom). Apart from this, the familiar unpleasant smell of flower water was substantially absent, because the bacterial decomposition processes were slowed down and delayed.

What is claimed is:

1. A method of providing a product with a bacteriostatic property, the method comprising:
(a) providing a product including a surface;
(b) providing a textile microbicidal repository material comprising fibers and a solid composite of silver-ion loaded ion-exchanger particles with a hydrophilic matrix;
(c) placing the textile material into an aqueous solution to cause the release of at least a portion of the silver ions from the textile material; and
(d) placing the product into the solution in the presence of the textile material for a predetermined period of time,
wherein the silver ions are transferred from the textile material to the surface of the product to provide the product with a bacteriostatic property.

2. The method according to claim 1, wherein (b) comprises (b.1) providing a textile microbicidal repository material including lyocell fibers and a solid composite of silver-ion loaded ion-exchanger particles with a cellulose matrix.

3. The method according to claim 1, wherein the textile material comprises one of a non-woven fabric, a warp-knitted fabric, and a textile woven fabric.

4. The method according to 1, wherein the textile microbicidal repository material further includes a water-permeable sheath.

5. The method according to claim 1, wherein the aqueous solution is generated by one of a laundry machine and a dish-washing machine.

6. The method according to claim 1, wherein the product is a plant or flower including a stem, and step (d) comprises (d.1.) inserting the stem into the aqueous solution.

7. The method according to claim 1, wherein the silver ions are present in the textile material repository in an amount of less than about 10% w/w.

8. The method according to claim 1, wherein an equilibrium concentration of the silver ions is maintained in a biologically effective range.

9. A method of laundering clothing comprising:
(a) providing a laundry machine;
(b) filling the laundry machine with water to form an aqueous solution;
(c) providing a textile microbicidal repository comprising fibers and a solid composite of silver-ion loaded ion-exchanger particles in a hydrophilic matrix;
(d) providing clothing;
(e) placing the textile repository into the aqueous solution to cause the release of at least a portion of the silver ions from the textile repository into the solution;
(f) placing the clothing into the aqueous solution in the presence of the textile repository; and
(g) agitating the clothing to generate the transfer of the silver ions from the textile repository to a surface of the clothing to provide the clothing with a bacteriostatic property.

10. The method according to claim 9, wherein (c) comprises (c.1) providing a textile microbicidal repository including lyocell fibers and a solid composite of silver-ion loaded ion-exchanger particles in a cellulose matrix.

11. The method according to claim 9, wherein the textile repository is one of a non-woven fabric, a warp-knitted fabric, and a textile woven fabric.

12. The method according to 9, wherein the textile microbicidal repository further comprises a water-permeable sheath.

13. A method of treating a fresh plant or flower comprising:
(a) providing a plant or flower including a surface;
(b) providing a textile microbicidal repository comprising fibers and a solid composite of silver-ion loaded ion-exchanger particles with a hydrophilic matrix;
(c) placing the textile repository into an aqueous solution to cause the release of at least a portion of the silver ions from the textile repository; and
(d) placing the flower or plant into the aqueous solution in the presence of the textile repository into the solution,
wherein the silver ions is released by the textile repository are transferred from the textile repository to the surface of the plant or flower in an amount effective to inhibit the formation of bacteria that causes the decomposition of the flower or plant.

14. The method according to claim 13, wherein the silver ions are effective to inhibit the visible withering and drooping of the flower or plant compared to a solution not including the silver ions.

15. The method according to claim 13, wherein (b) comprises (b.1) providing a textile microbicidal repository including lyocell fibers and a solid composite of silver-ion loaded ion-exchanger particles with a cellulose matrix.

16. The method according to claim 13, wherein the textile repository comprises one of a non-woven fabric, a warp-knitted fabric, and a textile woven fabric.

17. The method according to 13, wherein the textile microbicidal repository further comprises a water-permeable sheath.
